(19) United States
(12) Patent Application Publication
(10) Pub. No.: US 2025/0152449 A1
(43) Pub. Date: May 15, 2025

(54) SPHERICALLY COUPLED, CUSTOMIZED, DYNAMICAL TRANSARTICULAR, ANATOMICAL EXTERNAL FIXATOR

(71) Applicants: Fabrizio Granieri, Trani (BT) (IT); Giuseppe Giannicola, Roma (IT)

(72) Inventors: Fabrizio Granieri, Trani (BT) (IT); Giuseppe Giannicola, Roma (IT)

(21) Appl. No.: 18/835,665

(22) PCT Filed: Feb. 9, 2023
(86) PCT No.: PCT/IB2023/051162

§ 371 Date: Aug. 2, 2024

(30) Foreign Application Priority Data
Feb. 15, 2022 (IT) 102022000002708

(51) Int. Cl. A61H 1/02

(52) U.S. Cl. CPC A61H 1/0277; A61H 2201/0192; A61H 2201/1676; A61H 2201/5007; A61H 2201/5084; A61H 2201/5092

(57) ABSTRACT

A spherically coupled customized dynamical transarticular anatomical external fixator designed for the rehabilitation of human body joints, particularly the elbow, but also applicable to the knee, shoulder, wrist, ankle, hip, and others. This device includes a first portion with fixation means to secure a rigid bracket to a bone of the limb's proximal portion and a second portion with fixation means to secure another rigid bracket to a bone of the distal portion. A spherical shell with projections is fixed to one bracket, while a shaped sliding guide with tracks is fixed to the other, enabling only the physiological movement of roto-translation and flex-extension of the joint. This precise movement is detected during a patient's specific articulation and is essential for effective rehabilitation, ensuring movements are natural and prevent joint damage.

### BACKGROUND OF THE INVENTION

### 1. Field of the Invention

Object of the present invention is an orthopedic (articulated), dynamical, transarticular, anatomical external fixator intended for surgical treatment and rehabilitation of human body articulations, in particular elbow but also knee, shoulder, wrist, ankle, hip and other articulations.

### 2. Brief Description of the Prior Art

It is known that after surgery or a trauma, in order to restore the correct functionality of an articulation, an early articular mobilization in the physiological planes of movement is needed. In particular, in case of elbow articulation rehabilitation, repeated flexion and extension exercises combined with prone/supination movements of the forearm are also needed. So, at the state of the art, various machines are known that allow this kind of movements to be carried out in a guided manner, both passively (with the machine carrying out the exercise by means of a motorized system) and actively (with the patient who has to exert the strength needed to carry out the exercise).

By analyzing, for simplicity, the case of the elbow rehabilitation, at the state of the art there are known many examples of rehabilitation devices configured to be fastened to the patient arm and forearm and to allow arm flexion and extension movements to be carried out in a guided manner. Without claiming to be exhaustive, some examples are described in CN103251493, CN103536426, CN103976852A, MI2010A001769.

Anyway, these and other devices known at the state of the art are limited since their kinematism does not allow to make the center of instantaneous rotation of the articulation coincide constantly with the center of instantaneous rotation of the kinematism during the whole extension movement, and also because they eliminate completely the possibility of forearm rotation around its own axis (prone/supination movement).

Some devices known at the state of the art combine movements of flex-extension and prone-supination, but they are not able to impose the execution of only one complex trajectory and are not able to follow correctly the effective center of instantaneous rotation of the articulation, so during their usage they can ruin the articulation itself by making it carry out a movement other than the natural one.

Ultimately, such devices do not allow to individuate and carry out a unique complex movement useful for rehabilitation and recovery.

One of the current inventors has yet contributed to this specific technical field, by inventing a device for articular rehabilitation comprising a brace provided with a joint with spherical hinge, described in WO2020/152549. Anyway, also the device described in this document, even if very useful in managing rehabilitation and recovery of subjects with articular traumas, is limited since the joint with spherical hinge involves a constant relative position of the center of rotation of the proximal and distal portion of a limb, and its displacement is carried out only by the translation of the telescopic rods which make the CIR of the device follow the CIR of the articulation only in the longitudinal direction which is greater, instead the translations in the two transverse directions, which are smaller, are absorbed by the muscle and supports.

Such device favors all the possible articulation movements, making the center of instantaneous rotation of the articulation coincide with the center of instantaneous of the brace, without imposing a particular trajectory in the passive version of the device, i.e. without actuators.

By means of the device described therein, it is impossible to identify the complex trajectory describing a movement individuated as correct for the articulation, and to make the articulation itself carry out only that movement.

It is in fact known that the respective movement of the distal portion of a limb (forearm) relative to the proximal portion thereof (arm) is a complex combination of rotations and translations, that the whole articulation (when healthy) obliges the two portions to move according to complex trajectories. So, during rehabilitation, it is needed that the function of imposing only one trajectory of correct movement is carried out by an external brace.

To the best of the current inventor's knowledge, no one of the devices known at the state of the art allows to identify the correct trajectory of the relative movement between the distal and proximal portion of a limb for the specific patient and so to impose the possibility to carry out only that movement.

This is due to the anatomical variants of the articulations of the population. All the current commercialized devices, called dynamical transarticular external fixators, do not consider this variability among individuals, and so impose a not physiological articular trajectory, since it is realized as an average of the trajectories of the many individuals.

Therefore, aim of the present invention is to provide a customizable external fixator for rehabilitation of human body articulations, in particular the elbow but also the knee (and other articulations), which overcomes the limits linked to the embodiments known at the state of the art, and which, in particular, allows to identify the correct trajectory of the relative movement between the distal and proximal portion of a limb, physiologically followed by each specific patient and to limit, during usage, the movements allowed only to that movement.

Specifically, the fixator according to the invention, by allowing intra-operative acquisition of data configuring the exact patient's kinematics, is able to allow in post-operative step to move the articulation along its movement planes neutralizing effectively all the cutting, compression, distraction, dislocation forces which would determine luxation of articulation or damages to damaged, repaired and in recovery step articular structures.

Ultimately, the main therapeutical indications of such a device are: articular and periarticular fractures, luxations, fractures-luxations, chronicle instabilities, articular rigidities, articular deformities, pseudoarthrosis and all the other articular and periarticular diseases requiring a dynamical transarticular fixation during treatment.

### SUMMARY OF THE INVENTION

The invention realizes the prefixed aims since it is a spherically coupled customized dynamical transarticular anatomical external fixator for the rehabilitation of human body articulations, comprising a first portion (1) and a second portion (2) movable to each other, said first portion (1) comprising fixation means (11) configured to fix a first rigid bracket (12) to at least a bone (100) of the proximal portion of a limb, said second portion (2) comprising fixation means (21) configured to fix a second rigid bracket (22) to at least a bone (200) of the distal portion of a limb, to one of said first (12) and second rigid bracket (13) a spherical shell portion (13) being fixed, which is sized for receiving the articulation considered therein, said spherical shell portion (13) being provided with a plurality of outer projections (131), to the other one of said first (12) and second rigid bracket (22) a shaped sliding guide (23) being fixed, which is provided with a plurality of sliding tracks (231), characterized in that said shaped sliding guide (23) and said tracks (231) are shaped for respectively coupling to said spherical shell portion (13) and to said projections (131) so that said spherical shell portion (13) is allowed only to carry out a movement of roto-translation and flex-extension with respect to said shaped sliding guide (23) coincident with the physiological movement of roto-translation and flex-extension of the articulation.

### BRIEF DESCRIPTION OF THE DRAWINGS

FIGS. 1 and 2 show two schematic views of the device according to the invention and of the bones of arm and forearm;

FIG. 3 shows a schematic view of the device without indication of the presence of the articulation bones;

FIG. 4 shows a schematic view of the sphere provided with projections;

FIGS. 5 and 6 show two schematic views of the device according to the invention and of the bones of arm and forearm in trajectory detection step, with support for detection sensors installed;

FIGS. 7 and 8 show two views respectively illustrating the portion of the device which, during the exercise, is fastened to the distal portion of the limb and the portion of the device, which during the exercise, is fastened to the proximal portion of the limb.

FIG. 9 shows an exemplary image of the tracks provided on the inner surface of the outer shell.

FIG. 10 shows an exemplary image of another embodiment of the device.

FIGS. 11 and 12 show two views of the device in a configuration in which the fastening occurs by means of outer supports and not by means of orthopedic screws.

FIGS. 13 to 16 show an embodiment using an electrogoniometer. FIG. 13 shows an embodiment in which the spherical shell is fastened to the distal portion of the limb.

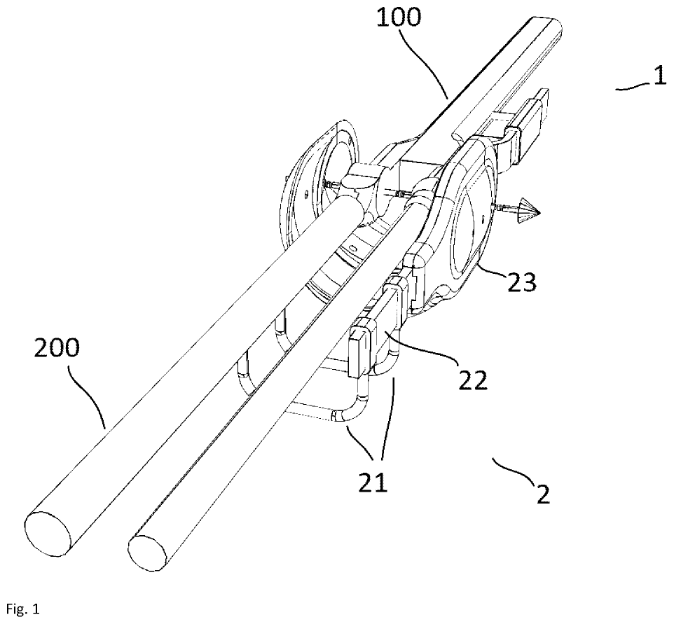

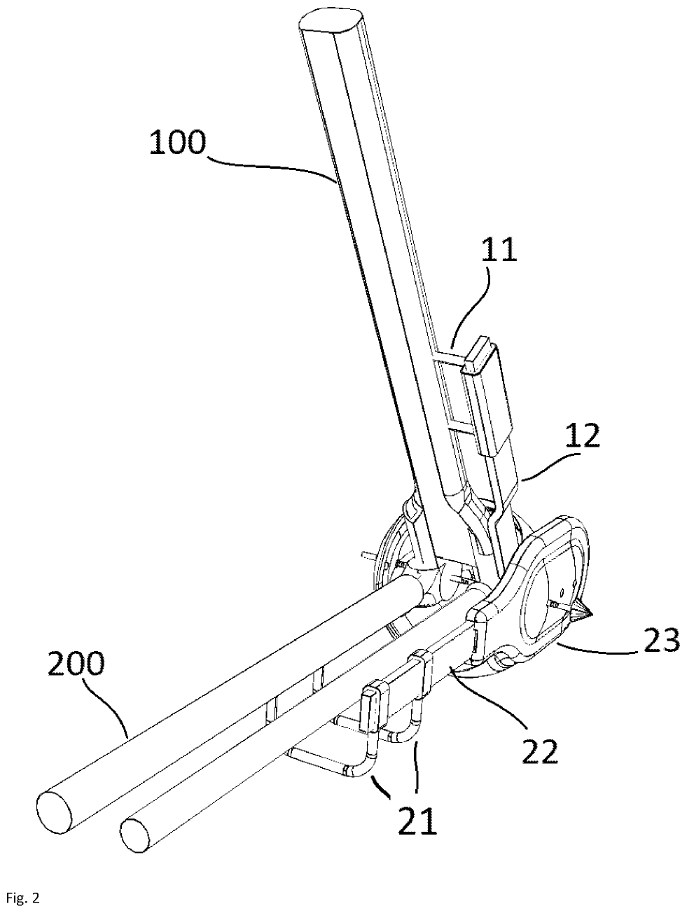

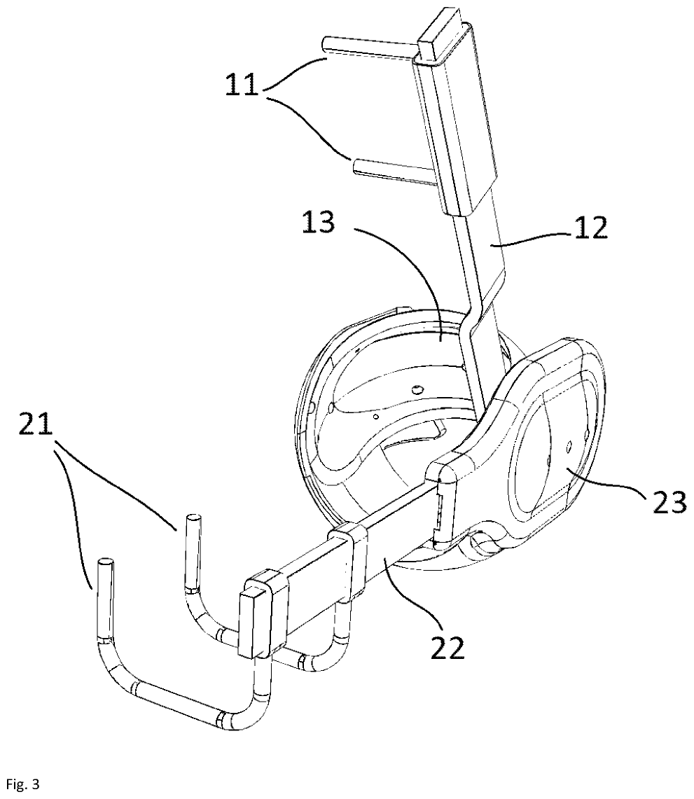

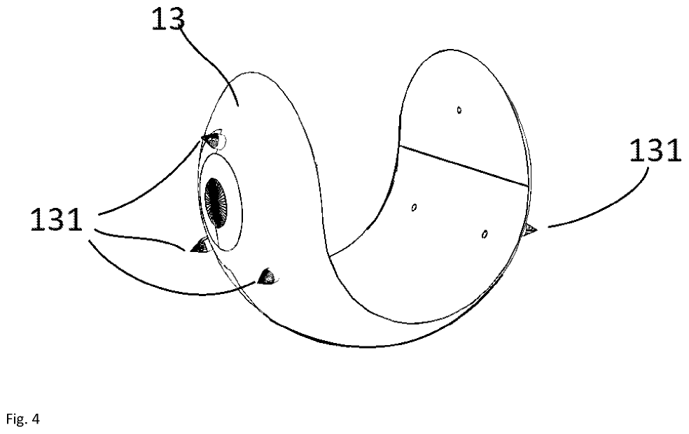

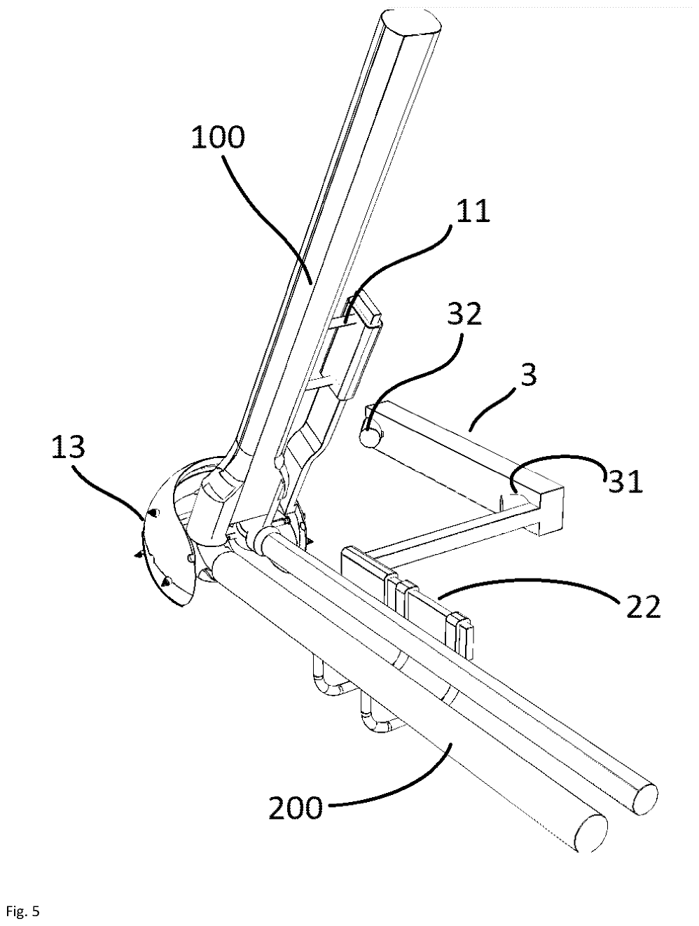

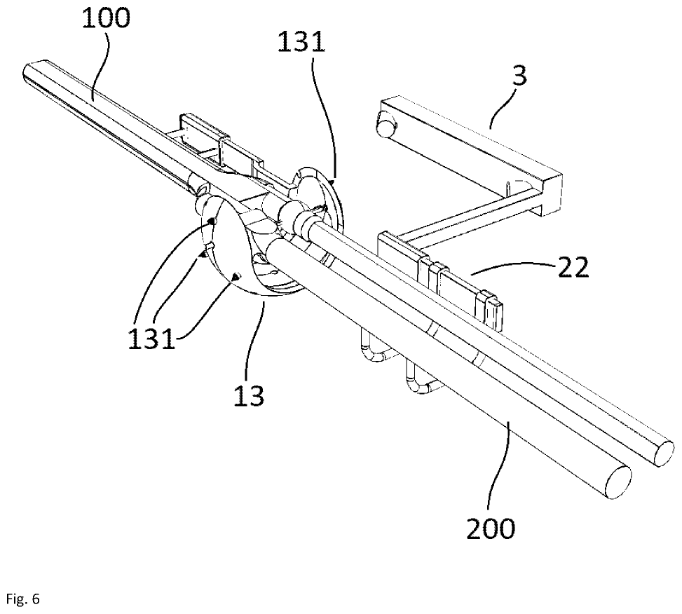

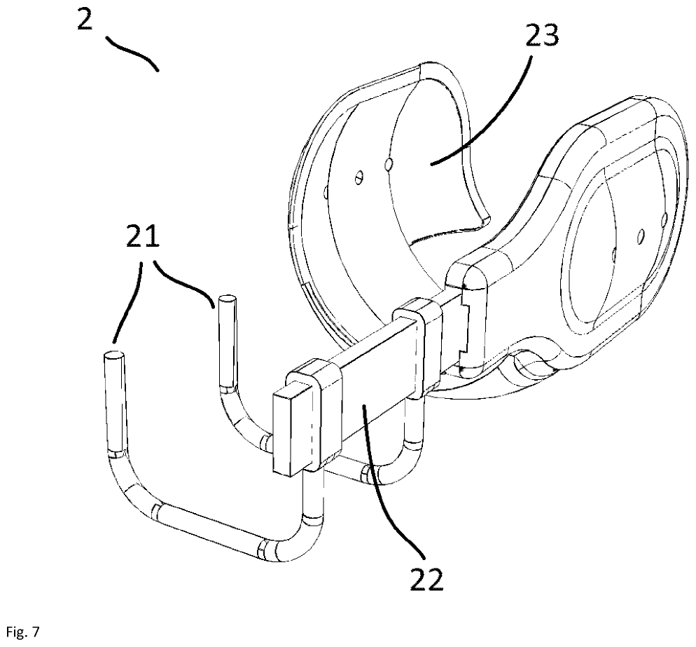

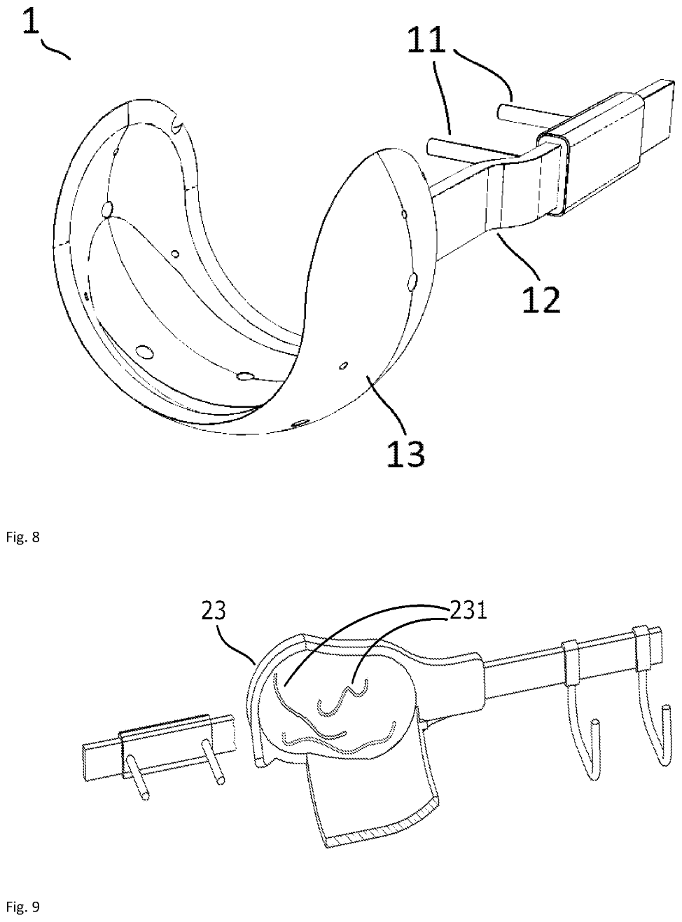

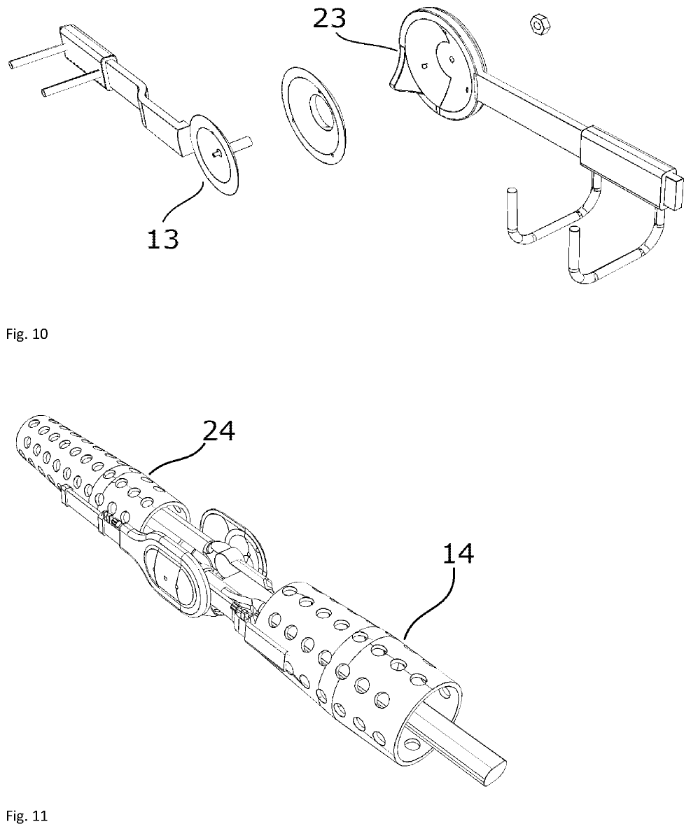

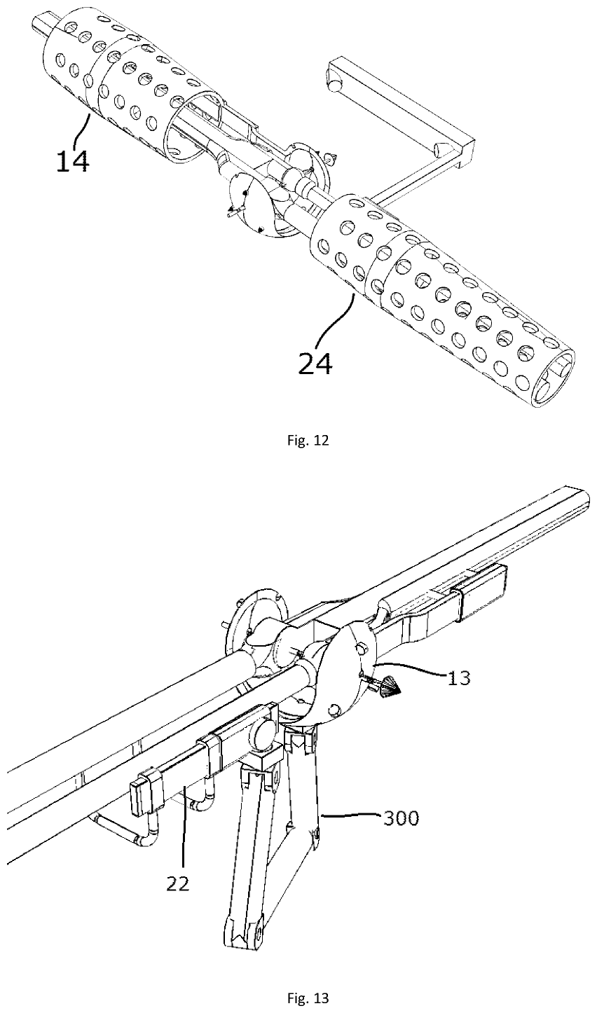

### DETAILED DESCRIPTION OF THE PREFERRED EMBODIMENTS

With reference to the appended drawings, the device according to the invention comprises a first portion (1) and a second portion (2).

The first portion (1) comprises fixation means (11) configured to fix a first rigid bracket (12) to at least a bone (200) of the distal portion of a limb. The fixation means (11, 12) are preferably orthopedic screws (typically of fiches type or Schanz screws), whose fixation to a bone is known per se at the state of the art. To the rigid bracket (12)—which, in the commercialized devices is commonly called “body of the fixator”—a spherical shell portion (13) is fixed, provided with a plurality of projections (131). Once the bracket (12) and the shell (13) are fixed, no residual degrees of freedom are available, and the spherical shell is rigidly fixed to the distal portion of the limb (to the ulna in case the device is used for the elbow articulation).

Without departing from the aims of the invention, the portion with the spherical shell (13) can be fastened to the proximal portion and the portion with the guide can be fastened to the distal portion.

It is highlighted that the described device, as it will be clear from the following detailed description, is particularly indicated for the early mobilization (from early after surgery) of the articulations, by safeguarding the articular congruity and preserving the reduction and osteosynthesis of articular fractures, in addition to the reparation or reconstruction of capsule-ligament structures. Ultimately, it is useful to protect the recovery of bone, capsule-ligament and tendinous tissues during articulation early mobilization.

It is also to be precised that between the screws and the brackets a position adjustment system is preferably provided, known per se at the state of the art and not represented in the appended figures, which allows to adjust the relative position of the bracket to the bone it is fixed to. In this way, it is possible to install the brackets with axis coincident with the longitudinal extension of arm and forearm.

It is to be specified that the spherical shell portion (13) is sized for receiving the articulation considered therein, i.e. the articulation of the elbow, by positioning itself at the back of the arm, or the articulation of the knee, by positioning itself frontally to the leg, or the articulation of shoulder or other articulations. Yet, it is to be specified that the term spherical refers to the outer surface of the shell (13), which will be coupled to the second portion of the device, according to what described in the following.

It is also to be specified that the projections (131) are elements (such, for example pins with cylindrical section) integral to the spherical shell (13) and projecting to the outer surface of the same. Preferably, said elements project in radial direction to the outer surface of the sphere, and have similar size, so that their tips are in turn positioned on a spherical surface. In any case, since the geometry of the spherical shell (13) is known, the tip position of each projection to the center of the sphere is known.

The second portion (2) comprises fixation means (21) configured to fix a second rigid bracket (22) to at least a bone (200) of the proximal portion of a limb. The fixation means (21) are preferably orthopedic screws, whose fixation to a bone is known per se at the state of the art. To the rigid bracket (22) a shaped sliding guide (23) is fixed, provided with a plurality of sliding tracks (231). Once the bracket (22) and the shaped sliding guide (23) are fixed, no residual degrees of freedom are available, and the guide is rigidly fixed to the distal portion of the limb (to the ulna in case the device is used for the elbow articulation).

The device is characterized in that said shaped sliding guide (23) is shaped for coupling to said spherical shell portion (13) and to said projections (131) so that said spherical shell portion (13) is allowed to carry out only the roto-translational movement to said shaped sliding guide (23) coincident with the physiological roto-translational movement of the articulation chosen for rehabilitation and recovery.

Such movement, as better explained in the following, is detected during execution of the movement by each specific patient to whom the spherical shell portion (13), installed integrally to the distal portion of the limb, and a detection system in the three space coordinates, installed integrally to the proximal portion of the limb, have been applied yet.

It is clear that, since it is needed to detect a relative movement, the device can also provide a spherical shell portion associated to the proximal portion of the limb and a shaped guide associated to the distal portion, without departing from the aims of the invention.

The trajectory associated to the articular movement carried out by each specific patient is a complex trajectory, composed by a series of relative rotations and translations of the forearm to the arm (or the leg to the thigh) interesting all the six possible degrees of freedom (rotations around three axes orthogonal to each other, translations along three axes orthogonal to each other). It is to be specified in particular that the trajectory of the physiological movement is the detected one, without brace/fixator, for the specific patient, by means of execution-possibly guided by healthcare staff—of the relative movement.

Ultimately, in a first functioning step the articulation can carry out any movement among the physiological ones for the same. It is chosen one thereof, which is detected by the device. So, the detected movement will be the only possible one which will be carried out in the following when the device is applied to the patient.

More in particular, the inner surface of the shaped guide (23) is provided with a shape corresponding to the envelope of all the positions progressively taken by the outer surface of the spherical shell portion (13) during the physiological movement of the articulation.

Furthermore, on the surface of the shaped guide (23), a plurality of sliding tracks or guides are provided, associated each to a respective projection (131) provided on the spherical shell portion (13) and corresponding each to the trajectory covered by the relative projection (131) during execution of the movement by the patient to whom the spherical shell portion has been applied integrally to the distal portion of the limb.

It is clear that the difference between the fixator, object of the present invention, and all the other fixators known in the market is that neither the execution of a simple rotation (as it occurs in case of a joint with cylindrical hinge), nor the execution of a composed rotation (as it occurs for joints with spherical hinge) and the execution of a predetermined roto-translational movement (regardless of its complexity) is allowed. On the contrary, the fixator, object of the present invention, allows only the execution of the effective roto-translational physiological movement carried out by the specific articulation of the specific patient.

It is now described a preferred embodiment of the device, and in particular of the geometry of the shaped guide (23) which allows the execution of the suitable movement for each specific patient. For simplicity of description, the device is described associated to the elbow, and so it will be referred to the humerus and ulna as interested bones. The device can be used similarly for the articulation of knee or for other articulations.

In a first step, the orthopedic screws (11) are fixed to the ulna (100), and, on the same, a respective bracket (12) and the spherical shell portion (13) are fixed with the relative projections (131).

The relative orthopedic screws (21) are fixed to the humerus (200), and on a rigid bracket, to said fixators (21) associated to the humerus (200) a vision system (3) is rigidly associated comprising at least two position detection means (31, 32).

Preferably, said position detection means are at least two cameras.

It is observed that in this step the position of the two cameras (31, 32) to the fixators (21) is determined univocally.

Clearly, the visors can be fastened to the ulna, and the spherical shell portion can be fastened to the humerus, without departing from the aims of the invention.

It is also to be observed that once known, because it is detected by means of the vision system, the position of at least three points integral to the spherical shell (13) in the reference system integral to the ulna (200) (or in the reference system integral to the humerus, in case of spherical shell fastened to the ulna), it is known both the position of the center of the spherical shell portion (13), and the position of all the projections (131), whose geometrical configuration is known. In a first embodiment, the three points considered are relative to the tips of three different projections (131). In another configuration, the three points considered are relative to colored markers provided on the spherical shell and/or projections (131).

So, the detection is carried out by acquiring a plurality of consecutive photograms by means of said vision system (3) and by identifying in each of them the position of at least three points integral to said spherical shell portion or said projections (131).

In only one 3D acquisition it is detected also the tridimensional trajectory (composed by arbitrary translations and rotations in the space) carried out by the spherical shell portion (13) in the reference system integral to the ulna (200) while the articulation realizes its own physiological movement which is desired to the replicated.

According to the conditions of the patient, such movement can be guided by the orthopedist or physiatrist, or the patient can carry out it by oneself if he is able to.

Possibly, the movement can be carried out also with “open” articulation during surgery, so that the orthopedist can see the elbow articulation; as an alternative, the movement is carried out with “closed” articulation.

Such acquired movement can be also corrected according to the specifics of the orthopedist or physiatrist in order to provide a correction of the articulation and so the portion (23) can be also realized so that a deviation to the acquired movement is introduced. According to another embodiment, the device can also act as distractor. In this case, the trajectory acquired by the 3D scanner is modified so to distance the ulna from the humerus of few millimeters-typically 1 or 2 mm and however lower than 5 mm—and so to allow a lower charge of the articulation in the movements.

In another embodiment, the functioning as distractor can be obtained by providing at least one of said first and second rigid bracket with a telescopic system and a relative blocking system.

In this way, once the system is arranged on the patient, it will be possible to elongate one or both the rigid brackets of a predetermined length (for example 1 mm), thus obtaining the functioning as distractor.

Preferably, but not limitingly, between the brackets and the orthopedic screws there are also applied charge cells, in order to measure the forces exchanged between the brackets and the bones and to check if they are comprised in acceptable value ranges.

In this way, by means of the vision system (3), the trajectory is acquired, which is followed by each of the at least three points individuated in each acquisition photogram, and so the movement carried out by the spherical shell portion (13) and by the various projections (131) can be reconstrued, which will be generally corresponding to a combination of movements of flex-extension, rotation and translation of the limb.

Since such a movement is acquired, it is possible to model the shaped surface (23) tri-dimensionally, corresponding to the envelope of all the positions progressively taken by the outer surface of the spherical shell portion (13) on which a plurality of sliding tracks or guides are realized, associated each to the trajectory covered by a respective projection (131). Once the shaped surface (23) is modelled, the same can be realized physically.

It is to be specified that at the state of the art, a plurality of tridimensional modelling software and a plurality of technologies for the realization of mechanical pieces are available, which can be conveniently used without departing from the aims of the present invention.

Conceptually, the shaped surface (23) is realized by subtraction of all the volumes progressively associated to the track acquired by the 3D scanner from a full volume.

For example, the shaped guide (23) can be realized by means of rapid prototyping, sintering or by chip removal according to any technique known per se, known at the state of the art. The shaped guide (23) in integral to a respective bracket (22) integral to the orthopedic screws (21). The bracket is sized for ensuring the correct positioning of the shaped guide (23) in the reference system integral to the ulna (200).

At this point, the device is completed, and the only movement allowed to the articulation is exactly the one detected for the specific patient with possible corrections decided by the orthopedist or physiatrist.

So, it is possible to proceed to check if the articulation moves correctly by carrying out the movement (possibly in assisted way) and by measuring the forces exchanged between the orthopedic screws and brackets by means of charge sensors.

Without this being limiting for the aims of the invention, it is possible to install at least an actuator, linear or of any other type, between the bracket (12) integral to the proximal portion of the limb and the bracket (22) integral to the distal portion of the limb. The elongation or retraction of the linear actuator will imply the relative movement of the distal portion of the limb to the distal portion, which obviously can occur only along the only trajectory allowed by the device.

In this way, it is obtained the possibility to carry out guided rehabilitation exercises with a movement precision that it is not allowed by any other device among the ones known at the state of the art. Furthermore, the device allows to adjust the articular excursion desired and to increase or decrease it according to the needs.

FIG. 10 shows an exemplary image of another embodiment of the device, in which the spherical shell portion (13) and the shaped guide (23) are arranged only on a side of the articulation, and are fixed to each other for example—by means of a threaded pin integral to the spherical shell portion, passing in a hole provided in the shaped guide and fixed by means of a clamping nut.

In another embodiment, instead of fixing with orthopedic screws, in order not to carry out an invasive procedure, the brackets of the device can be fastened to a couple of supports (14, 24), possibly realized by means of a scan of the geometry of the arm and forearm, which are fastened respectively to the arm and forearm of the patient. Even if the supports act on soft tissues—and so their fastening is not very correct—this aspect avoids anyway the introduction of Fiches in the bone, and so no surgery or invasive procedure has to be carried out.

In another embodiment, the detection system does not comprise a vision system, but it provides, on the spherical shell portion, tracing elements (marker pens, chalks, other) configured to sign traces relative to its own path on the inner surface of the outer guide. The traces drawn by said tracing elements on said inner surface are so worked by chip removal to realize tracks (231), whose functioning has been described yet.

In the light of what explained it is observed that, in order to have the device to be applied to the patient immediately available at the moment of surgery, the device can be realized in a plurality of standard sizes and dimensions.

This occurs by detecting the roto-translational physiological movement carried out by a patient of whom a set of anthropometrical data are known, comprising at least and in particular the length of the bones of arm and forearm, measured from the attachment point of the screws at the center of said spherical shell portion, and by associating said anthropometrical values to the geometry obtained for the shaped sliding guide.

By carrying out the detection for a plurality of subjects with different anthropometrical data, a database of geometries is obtained for the shaped guide, each one associated to a corresponding set of anthropometrical data.

In this way, it is possible to avoid the detection step of the geometry for patients whose anthropometrical data are similar (i. e. similar except for a predefined tolerance) to the ones of a patient for whom the geometry of the shaped guide has been detected.

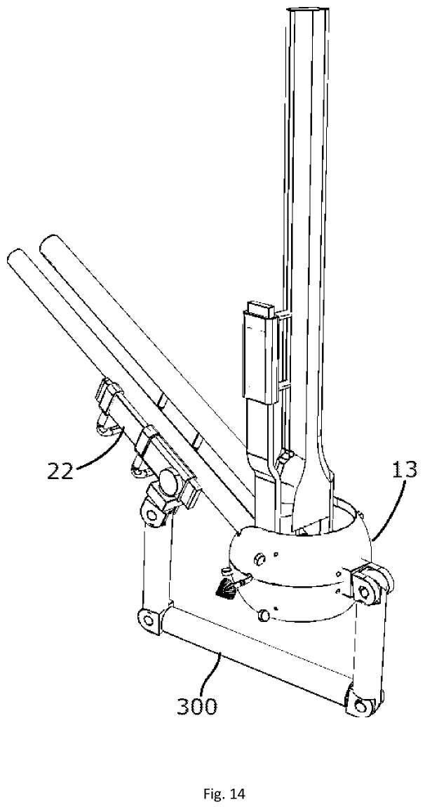

This allows to realize the shaped guides for usage with the device in a finite number of configurations, each one associated to a relative set of anthropometrical data, to the advantage of the fabrication affordability of the same guides. According to another embodiment shown in FIGS. 13 and 14, the detection of the roto-translational physiological movement can be carried out by using a digital electrogoniometer (300) with at least six degrees of freedom, fastened to the spherical shell portion (13) and to the brackets (22) fixed to the bones of the forearm.

The electrogoniometer is configured to detect and store the displacements and rotations of the spherical shell portion during relative movement between arm and forearm.

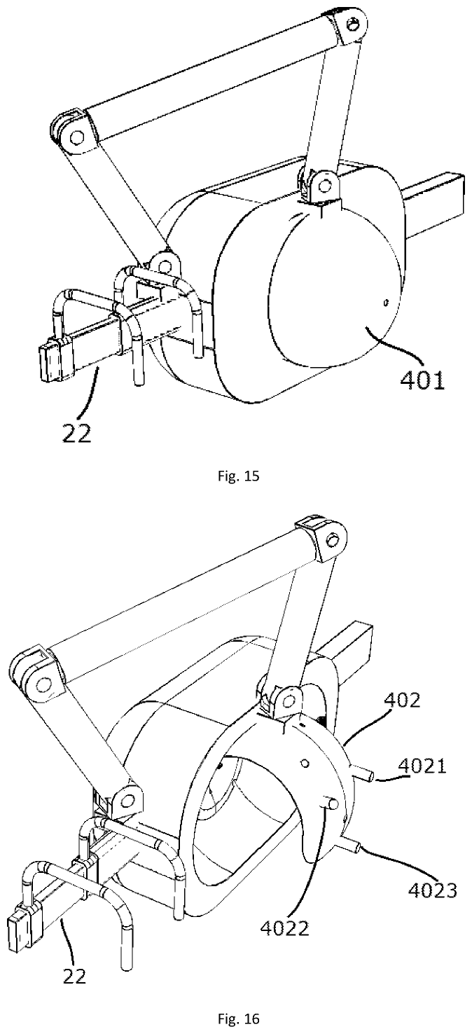

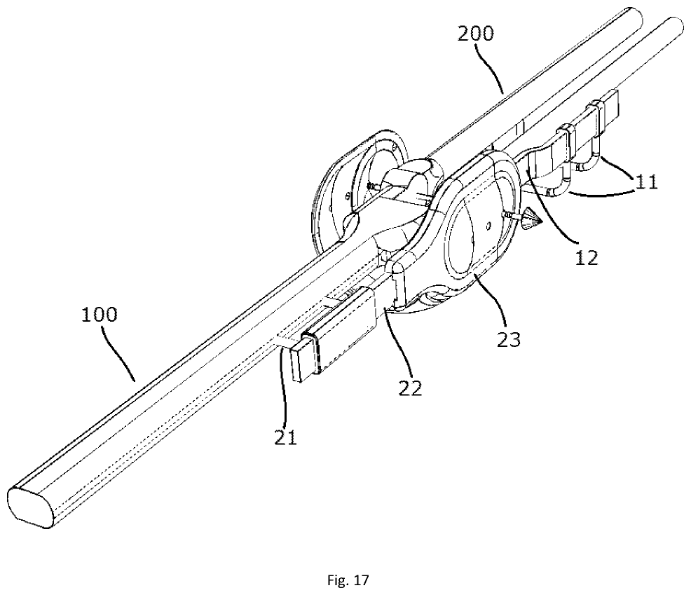

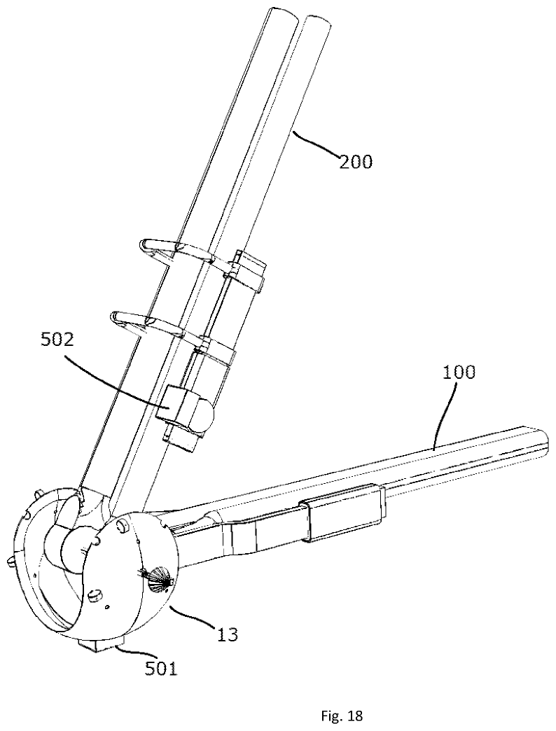

The relative movement (displacements and rotations) stored by means of the electrogoniometer is so used to realize the shaped guide by means of chip removal machining in two steps:
a first step (FIG. 15) in which a tool (401) of a numerical control machine realizes the shaped sliding guide by means of chip removal;
a second step (FIG. 16), in which a tool (402) provided with a plurality of working heads (4021, 4022, 4023) realizes the sliding tracks (231).

The working heads used in this second step are positioned in the same relative position as the projections provided on said spherical shell portion. Preferably, the numerical control machine used in this working step has the same geometry, in terms of arms and joints, as the digital electrogoniometer, so that it is not needed a reprocessing of the acquired displacements and rotations.

It is to be specified that, without departing from the aims of the invention, the sliding tracks can be realized in a plurality of successive workings carried out by a tool with only one head.

In another embodiment, the acquisition of the movement of the distal portion of the limb to the proximal portion can be carried out by means of an acquisition system comprising two sensors (501, 502), each one comprising a gyroscope with three axes and an accelerometer with three axes, the one (501) integral to the spherical shell portion (13) and the other one (502) integral to the brackets (22) fixed to the bones of the forearm.

The acquisition of the orientation and accelerations of the distal and proximal portion of the arm allows to reconstruct the tridimensional trajectory (composed by arbitrary translations and rotations in the space) carried out by the spherical shell portion (13) (which is preferably integral to the ulna) in the reference system integral to the humerus, while the articulation realizes its own physiological movement which is desired to be replicated.

1. A spherically coupled, customized, dynamical, transarticular, anatomical external fixator for the rehabilitation of human body articulations, comprising a first portion (1) and a second portion (2) movable to each other,
wherein said first portion (1) comprising fixation means (11) configured to attach a first rigid bracket (12) to at least a bone (100) of the proximal portion of a limb,
wherein said second portion (2) comprising fixation means (21) configured to attach a second rigid bracket (22) to at least a bone (200) of the distal portion of a limb, to one of said first (12) and second rigid bracket (13) a spherical shell portion (13) being fixed, which is sized for receiving the articulation considered therein, said spherical shell portion (13) being provided with a plurality of outer projections (131), to the other one of said first (12) and second rigid bracket (22) a shaped sliding guide (23) being fixed, which is provided with a plurality of sliding tracks (231),
and wherein said shaped sliding guide (23) and said tracks (231) are shaped for respectively coupling to said spherical shell portion (13) and to said projections (131) so that said spherical shell portion (13) is allowed only to carry out a movement of roto-translation and flex-extension with respect to said shaped sliding guide (23) coincident with the physiological movement of roto-translation and flex-extension of the articulation.

2. The mobilization orthopedic fixator for articulation rehabilitation according to claim 1, wherein said physiological movement of roto-translation and flex-extension is detected during the execution by a patient to whom said spherical shell portion (13) has been yet applied integrally to the distal portion of the limb, by means of a detection system installed integrally to the proximal portion of the limb.

3. The mobilization orthopedic fixator for articulation rehabilitation according to claim 2, wherein said movement comprises a series of relative rotations and translations of the forearm to the arm, which affect all six possible degrees of freedom.

4. The mobilization orthopedic fixator for articulation rehabilitation according to claim 1, wherein the inner surface of the shaped guide (23) is provided with a shape corresponding to the envelope of all the positions progressively taken by the outer surface of the spherical shell portion (13) during the physiological movement of the articulation, and in that each one of said sliding tracks (231) has a profile corresponding to the trajectory covered by a relative projection (131) during execution of the movement by the patient.

5. The mobilization orthopedic fixator for articulation rehabilitation according to claim 1, wherein the surface of said shaped guide (23) is detected by means of the following method:
attaching said fixators (11), to a respective bracket (12) and said spherical shell portion (13) with the relative projections (131) to the ulna (200);
attaching said fixators (21), to a rigid bracket and a vision system (3) comprising at least two cameras to the humerus (100), so that the position of said cameras (31, 32) to said fixators (21) is univocally determined,
executing a physiological movement of the articulation, and detecting by means of said vision system of the tridimensional movement carried out by said spherical shell portion (13) in the reference system integral to the humerus (100),
tridimensional modelling and addition of possible corrections of said shaped surface (23) and said guides (231), defined respectively by the envelope of all the positions progressively taken during the movement of said outer surface of said spherical shell portion (13) and said projections (131).

6. The mobilization orthopedic fixator for articulation rehabilitation according to claim 5, wherein said detection is carried out by acquiring a plurality of consecutive photograms by means of said vision system (3) and by identifying in each one of them the position of at least three points integral to said spherical shell portion or to said projections (131).

7. The mobilization orthopedic fixator for articulation rehabilitation according to claim 5, wherein said shaped surface (23) is made, after its modelling, by means of rapid prototyping, sintering or by chip removal, and wherein, after its making it is installed integrally to said fixators (21) by means of a bracket (22) sized for ensuring the correct positioning of the shaped guide (23) in the reference system integral to the humerus (100), and coupled outside said spherical shell portion (13).

8. The mobilization orthopedic fixator for articulation rehabilitation according to claim 1, further comprising at least an actuator between the bracket (12) integral to the proximal portion of the limb and the bracket (22) integral to the distal portion of the limb.

9. The mobilization orthopedic fixator for articulation rehabilitation according to claim 1, wherein said projections (131) project in radial direction to the outer surface of the sphere, and are configured so that their tips are in turn positioned on a spherical surface.

10. The mobilization orthopedic fixator for articulation rehabilitation according to claim 1, further comprising charge cells installed between the brackets and the fixators, configured to measure the forces exchanged between the brackets and the bones.

11. The mobilization orthopedic fixator according to claim 4, wherein said trajectory acquired by means of the 3D scanner is modified so to distance the ulna from the humerus of a distance lower than 5 millimeters during functioning.

12. The mobilization orthopedic fixator according to claim 1, wherein said at least one of first and second rigid bracket is provided with a telescopic system and relative blocking system.

13. The mobilization orthopedic fixator according to claim 1, wherein said roto-translational physiological movement is detected on a patient of whom a set of anthropometrical data is known, comprising at least the length of the bones of an arm and forearm, measured from the attachment point of the screws at the center of said spherical shell portion, and by associating said anthropometrical values to the geometry obtained for the making of said shaped sliding guide.

14. The mobilization orthopedic fixator according to claim 13, wherein said detection of the roto-translational physiological movement is carried out by using a digital electrogoniometer (300) with six degrees of freedom, fastened to the spherical shell portion (13) and to the brackets (22) fixed to the bones of the forearm, configured to detect and store the displacements and rotations of the spherical shell portion during the relative movement between arm and forearm.

15. The mobilization orthopedic fixator according to claim 14, wherein said displacements and rotations are used to make the shaped guide by means of a chip removal machining in two steps:
a first step, in which a tool (401) of a numerical control machine makes the shaped sliding guide by means of chip removal;
a second step, in which a tool (402) provided with a plurality of working heads (4021, 4022, 4023) makes the sliding tracks (231).

16. The mobilization orthopedic fixator according to claim 13, wherein the detection of the roto-translational physiological movement is carried out by means of an acquisition system comprising two sensors (501, 502), each one comprising a gyroscope with three axes and an accelerometer with three axes, the one (501) integral to the spherical shell portion (13) and the other one (502) integral to the brackets (22) fixed to the bones of the forearm.
